(19) United States
(12) Patent Application Publication
(10) Pub. No.: US 2021/0022985 A1
(43) Pub. Date: Jan. 28, 2021

(54) COPOLYMER COMPOSITION FOR PERSONAL CARE

(71) Applicants: Dow Silicones Corporation, Midland, MI (US); Dow France S.A.S., Saint-Denis (FR)

(72) Inventors: Bizhong Zhu, Midland, MI (US); Martin Grasmann, Midland, MI (US); Vinita Pandit, Midland, MI (US); Xavier Thomas, Famars (FR)

(21) Appl. No.: 16/334,584

(22) PCT Filed: Aug. 18, 2017
(86) PCT No.: PCT/US2017/047471

§ 371 Date: Mar. 19, 2019

Related U.S. Application Data
(60) Provisional application No. 62/396,336, filed on Sep. 19, 2016.

(30) Foreign Application Priority Data
Feb. 15, 2017 (EP) 17305167.3

(51) Int. Cl. A61K 8/898; C08G 18/61; C08G 18/73; C08G 18/75; C08G 18/76; A61K 8/04; A61K 8/06; A61Q 5/12; A61Q 5/02; A61Q 19/00

(52) U.S. Cl. CPC A61K 8/898; C08G 18/61; C08G 18/73; C08G 18/755; A61Q 19/00; A61K 8/04; A61K 8/06; A61Q 5/12; A61Q 5/02; C08G 18/7621

(57) ABSTRACT

A siloxane-urethane-urea copolymer is useful as a film forming agent. The copolymer can be added to personal care compositions, including skin care compositions and hair care compositions.

### CROSS REFERENCE TO RELATED APPLICATIONS

This application claims priority to U.S. Provisional Patent Application Ser. No. 62/396336 filed on 19 Sep. 2017 and European Patent Application Serial No. 17305167.3 filed on 15 Feb. 2017. U.S. Provisional Patent Application Ser. No. 62/396336 and European Patent Application Serial No. 17305167.3 are hereby incorporated by reference.

### TECHNICAL FIELD

A siloxane-urethane-urea copolymer is useful as a film forming agent. The copolymer can be added in personal care compositions, such as hair care compositions and skin care compositions.

### BACKGROUND

Silicone resins (such as MQ and MQ-T type resins) have been used as film forming agents in personal care compositions. These silicone resins may suffer from the drawback of providing insufficient abrasion resistance (resistance to wear off) to the personal care compositions, or in some other instances may be too brittle and develop cracks relatively easily. Polyurethanes are being used also for these applications. While they have better abrasion resistance, they can suffer from inferior water resistance and other disadvantages as compared with silicone resins. There is an industry need for improved film forming agents for use in personal care compositions.

### SUMMARY

This invention relates to a care composition comprising a siloxane-urethane-urea copolymer and a carrier to permit application to substrates such as skin, leather, hair, textiles, and/or other fibers.

This invention also provides a method of using the copolymer for treatment of such substrates. This invention further provides a method the copolymer as a film forming agent.

### DETAILED DESCRIPTION

A personal care composition comprises: (1) a siloxane-urethane-urea copolymer (A), and (2) a carrier that permits application. The personal care composition may further comprise one or both of (B) an organic polyol and (C) a reaction product of an organic polyisocyanate and an organic polyol. The siloxane-urethane urea copolymer and/or combination of said copolymer, and one or both of the organic polyisocyanate and/or reaction product may act as a film forming agent in the personal care composition.

Copolymer (A) is a siloxane-urethane-urea copolymer comprising units of formulae:

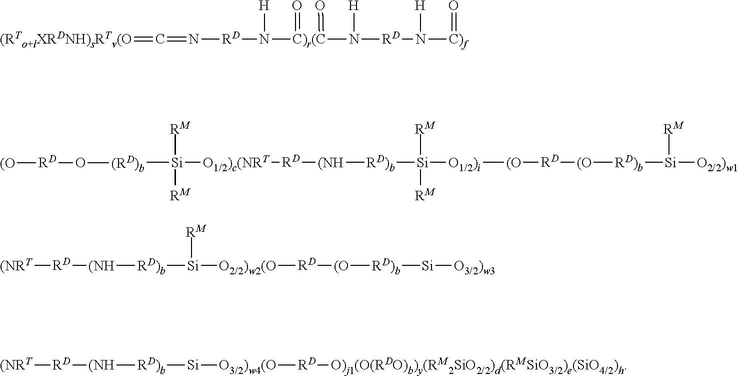

In the unit formula (A) above, each R^{D} is independently a divalent hydrocarbon group or a divalent halogenated hydrocarbon group, as defined below. Each R^{D} may independently have 2 to 13 carbon atoms. Alternatively, each R^{D} may be selected from alkylene such as ethylene or propylene, arylene such as phenylene, or alkaralkylene. Alternatively, each R^{D} may be an alkylene group such as ethylene or propylene.

Each R^{M} is independently a monovalent hydrocarbon group or a monovalent halogenated hydrocarbon group as defined below. Each R^{M} may have 1 to 13 carbon atoms. Alternatively, each R^{M} may be a monovalent hydrocarbon group free of aliphatic unsaturation. For example, each R^{M} may be independently selected from alkyl such as methyl, ethyl, propyl, butyl or hexyl; aryl such as phenyl, or aralkyl such as tolyl, xylyl or phenyl-methyl. Alternatively, each R^{M} may be methyl or phenyl, and alternatively each R^{M} may be methyl.

Each R^{T} is hydrogen or a monovalent hydrocarbon group. The monovalent hydrocarbon group for R^{T} may have 1 to 13 carbon atoms. The monovalent hydrocarbon group for R^{T} is group independently selected from alkyl such as methyl, ethyl, propyl, butyl, or hexyl; aryl such as phenyl; or aralkyl such as tolyl, xylyl, or phenyl-methyl. Alternatively, each R^{T} may be methyl or phenyl. Alternatively each R^{T} may be hydrogen or methyl.

Each subscript b is independently greater than or equal to 0. Each instance of subscript b can have a different value in a different unit of the copolymer. Alternatively, subscript b is 0 to 1,000,000. Alternatively, subscript b is 0 to 200,000. Alternatively, subscript b is 0 to 100,000. Alternatively, subscript b is 0 to 50,000. Alternatively, subscript b is 0 to 10,000. Alternatively, subscript b is 0 to 5,000. Alternatively, subscript b is 0 to 1,000. Alternatively, subscript b is 0 to 500. Alternatively, subscript b is 0 to 100. Alternatively, subscript b is 1 to 100. Alternatively, subscript b is 1 to 50. Alternatively, subscript b is 1 to 20. Alternatively, subscript b is 0 to 1. Alternatively, subscript b=1. Alternatively, subscript b=2. Alternatively, subscript b=3. Alternatively, subscript b=4. Alternatively, subscript b=5.

Subscript c≥0. Alternatively, subscript cis 0 to 200,000. Alternatively, subscript c is 0 to 100,000. Alternatively, subscript c is 0 to 50,000. Alternatively, subscript c is 0 to 10,000. Alternatively, subscript c is 0 to 5,000. Alternatively, subscript c is 0 to 1,000. Alternatively, subscript c is 0 to 500. Alternatively, subscript c is 0 to 100. Alternatively, subscript c is 0 to 50. Alternatively, subscript c is 0 to 20. Alternatively, subscript c is 0 to 10. Alternatively, subscript c is 1 to 100. Alternatively, subscript c is 1 to 50. Alternatively, subscript c is 1 to 20. Alternatively, subscript c is 1 to 10.

Subscript i≥0. Alternatively, subscript i is 0 to 200,000. Alternatively, subscript i is 0 to 100,000. Alternatively, subscript i is 0 to 50,000. Alternatively, subscript i is 0 to 10,000. Alternatively, subscript i is 0 to 5,000. Alternatively, subscript i is 0 to 1,000. Alternatively, subscript i is 0 to 500. Alternatively, subscript i is 0 to 100. Alternatively, subscript i is 0 to 50. Alternatively, subscript i is 0 to 20. Alternatively, subscript i is 0 to 10. Alternatively, subscript i is 1 to 100. Alternatively, subscript i is 1 to 50. Alternatively, subscript i is 1 to 20. Alternatively, subscript i is 1 to 10.

Subscript w1≥0. Alternatively, subscript w1 is 0 to 200,000. Alternatively, subscript w1 is 0 to 50,000. Alternatively, subscript w1 is 0 to 10,000. Alternatively, subscript w1 is 0 to 5,000. Alternatively, subscript w1 is 0 to 1,000. Alternatively, subscript w1 is 0 to 500. Alternatively, subscript w1 is 0 to 100. Alternatively, subscript w1 is 0 to 50. Alternatively, subscript w1 is 0 to 20. Alternatively, subscript w1 is 0 to 10. Alternatively, subscript w1 is 1 to 100. Alternatively, subscript w1 is 1 to 50. Alternatively, subscript w1 is 1 to 20. Alternatively, subscript w1 is 1 to 10.

Subscript w2≥0. Alternatively, subscript w2 is 0 to 200,000. Alternatively, subscript w2 is 0 to 50,000. Alternatively, subscript w2 is 0 to 10,000. Alternatively, subscript w2 is 0 to 5,000. Alternatively, subscript w2 is 0 to 1,000. Alternatively, subscript w2 is 0 to 500. Alternatively, subscript w2 is 0 to 100. Alternatively, subscript w2 is 0 to 50. Alternatively, subscript w2 is 0 to 20. Alternatively, subscript w2 is 0 to 10. Alternatively, subscript w2 is 1 to 100. Alternatively, subscript w2 is 1 to 50. Alternatively, subscript w2 is 1 to 20. Alternatively, subscript w2 is 1 to 10.

Subscript w3 0. Alternatively, subscript w3 is 0 to 200,000. Alternatively, subscript w3 is 0 to 50,000. Alternatively, subscript w3 is 0 to 10,000. Alternatively, subscript w3 is 0 to 5,000. Alternatively, subscript w3 is 0 to 1,000. Alternatively, subscript w3 is 0 to 500. Alternatively, subscript w3 is 0 to 100. Alternatively, subscript w3 is 0 to 50. Alternatively, subscript w3 is 0 to 20. Alternatively, subscript w3 is 0 to 10. Alternatively, subscript w3 is 1 to 100. Alternatively, subscript w3 is 1 to 50. Alternatively, subscript w3 is 1 to 20. Alternatively, subscript w3 is 1 to 10.

Subscript w4≥0. Alternatively, subscript w4 is 0 To 200,000. Alternatively, subscript w4 is 0 to 50,000. Alternatively, subscript w4 is 0 to 10,000. Alternatively, subscript w4 is 0 to 5,000. Alternatively, subscript w4 is 0 to 1,000. Alternatively, subscript w4 is 0 to 500. Alternatively, subscript w4 is 0 to 100. Alternatively, subscript w4 is 0 to 50. Alternatively, subscript w4 is 0 to 20. Alternatively, subscript w4 is 0 to 10. Alternatively, subscript w4 is 1 to 100. Alternatively, subscript w4 is 1 to 50. Alternatively, subscript w4 is 1 to 20. Alternatively, subscript w4 is 1 to 10.

A quantity (c+i+w1+w2+w3+w4) 1. Alternatively, in one embodiment i=w2=w4=0, and a quantity (c+w1+w3)≥1, for example, when the copolymer is prepared using a carbinol-functional polyorganosiloxane, as described below. In an alternative embodiment, c=w1=w3=0, and a quantity (i+w1+w3)≥1, for example, when the copolymer is prepared using an amine-functional polyorganosiloxane, as described below.

Each X is independently nitrogen (N), oxygen (O), or sulfur (S). Alternatively, X is N or O. Alternatively, each X is N. Alternatively, each X is O. Subscript o=0 when X is O or S, and subscript o=1 when X is N.

Subscripts d, e, and h depend on the molecular weight of one of the siloxane segments in the copolymer and may be without limit (e.g., bound only by the molecular weights reachable by the state of the art of siloxane synthesis chemistry). However, subscript d may be 0 to 1,000,000; subscript e may be 0 to 1,000,000; subscript h may be 0 to 1,000,000, and with the proviso that a quantity (d+e+h)≥1. Subscript d≥0. Alternatively, subscript d>0. Alternatively, subscript d is 0 to 200,000, alternatively 0 to 100,000, alternatively 0 to 50,000, alternatively 0 to 10,000, alternatively 0 to 5,000, alternatively 0 to 1,000, alternatively 1 to 1,000, alternatively 1 to 500, and alternatively 1 to 200.

Subscript e≥0. Alternatively, subscript e is 0 to 1,000,000. Alternatively, subscript e is 0 to 200,000, and alternatively 0 to 100,000, alternatively 0 to 50,000, alternatively 0 to 10,000, alternatively 0 to 5,000, alternatively 0 to 1,000, alternatively 1 to 1,000, alternatively 1 to 500, and alternatively 1 to 200. Alternatively, subscript e=0.

Subscript f indicates the number of urethane and/or urea units in the copolymer. Subscript f≥0. Alternatively, subscript f is 0 to 1,500,000. Alternatively, subscript f is 1 to 500,000, and alternatively 1 to 200,000, alternatively 1 to 50,000, alternatively 1 to 10,000, alternatively 1 to 5,000, alternatively 1 to 1,000, alternatively 1 to 500, and alternatively 1 to 200.

Subscript h is ≥0. Alternatively, subscript h is 0 to 1,000,000. Alternatively, subscript h is 0 to 200,000, and alternatively 0 to 100,000, alternatively 0 to 50,000, alternatively 0 to 10,000, alternatively 0 to 5,000, alternatively 0 to 1,000, alternatively 1 to 1,000, alternatively 1 to 500, and alternatively 1 to 200. Alternatively, subscript h=0.

Subscript j1 is ≥1. Alternatively, subscript j1 is >1 to 500,000. Alternatively, subscript j1 is >1 to 200,000, and alternatively 20 to 100,000, alternatively 50 to 50,000, alternatively 100 to 10,000, alternatively 1,000 to 5,000, alternatively 100 to 1,000, alternatively 10 to 500, and alternatively 15 to 200.

Subscript s is ≥0. Alternatively, subscript s is 0 to 200,000. Alternatively, subscript s is 0 to 150,000, and alternatively 0 to 100,000, alternatively 0 to 50,000, alternatively 1 to 10,000, alternatively 1 to 5,000, alternatively 1 to 1,000, alternatively 1 to 500, and alternatively 1 to 200.

Subscript v is ≥0. Alternatively, subscript v is 0 to 200,000. Alternatively, subscript v is 0 to 150,000, and alternatively 0 to 100,000, alternatively 0 to 50,000, alternatively 1 to 10,000, alternatively 1 to 5,000, alternatively 1 to 1,000, alternatively 1 to 500, and alternatively 1 to 200.

Subscript y is ≥0. Alternatively, subscript y is 0 to 200,000. Alternatively, subscript y is 0 to 150,000, and alternatively 0 to 100,000, alternatively 0 to 50,000, alternatively 1 to 10,000, alternatively 1 to 5,000, alternatively 1 to 1,000, alternatively 1 to 500, alternatively 1 to 200, alternatively 1 to 20, and alternatively 1.

Alternatively, when subscripts c=i=w2=w3=w4=e=h=0, copolymer (A) may have unit formula (I):

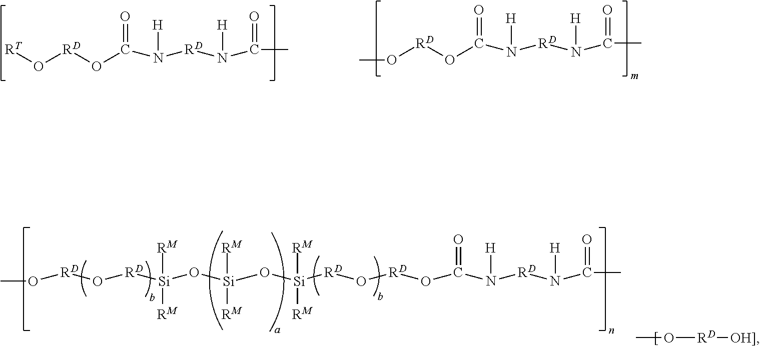

where R^{T}, R^{D} and R^{M} are as described above. Each subscript a is independently 0 to 1,000,000, each subscript m is independently greater than or equal to 0, each subscript b is independently greater than or equal to 0, and subscript n is greater than or equal to 1. Alternatively, each subscript b≥0. Alternatively, subscript b is 0 to 1,000,000. Alternatively, subscript b is 0 to 200,000. Alternatively, subscript b is 0 to 100,000. Alternatively, subscript b is 0 to 50,000. Alternatively, subscript b is 0 to 10,000. Alternatively, subscript b is 0 to 5,000. Alternatively, subscript b is 0 to 1,000. Alternatively, subscript b is 0 to 500. Alternatively, subscript b is 0 to 100. Alternatively, subscript b is 1 to 100. Alternatively, subscript b is 1 to 50. Alternatively, subscript b is 1 to 20. Alternatively, subscript b is 0 to 1. Alternatively, subscript b=0. Alternatively, subscript b=1. Alternatively, subscript b=2. Alternatively, subscript b=3. Alternatively, subscript b=4. Alternatively, subscript b=5.

Alternatively, copolymer (A) may have formula (II):

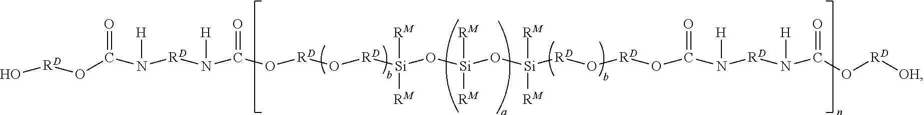

where R^{D} and R^{M} are as described above, subscript a is independently 0 to 1,000,000, each subscript b is independently greater than or equal to 0, and subscript n is greater than or equal to 1. Alternatively, each subscript b≥0. Alternatively, subscript b is 0 to 1,000,000. Alternatively, subscript b is 0 to 200,000. Alternatively, subscript b is 0 to 100,000. Alternatively, subscript b is 0 to 50,000. Alternatively, subscript b is 0 to 10,000. Alternatively, subscript b is 0 to 5,000. Alternatively, subscript b is 0 to 1,000. Alternatively, subscript b is 0 to 500. Alternatively, subscript b is 0 to 100. Alternatively, subscript b is 1 to 100. Alternatively, subscript b is 1 to 50. Alternatively, subscript b is 1 to 20. Alternatively, subscript b is 0 to 1. Alternatively, subscript b=0. Alternatively, subscript b=1. Alternatively, subscript b=2. Alternatively, subscript b=3. Alternatively, subscript b=4. Alternatively, subscript b=5.

Alternatively, copolymer (A) may have unit formula (III):

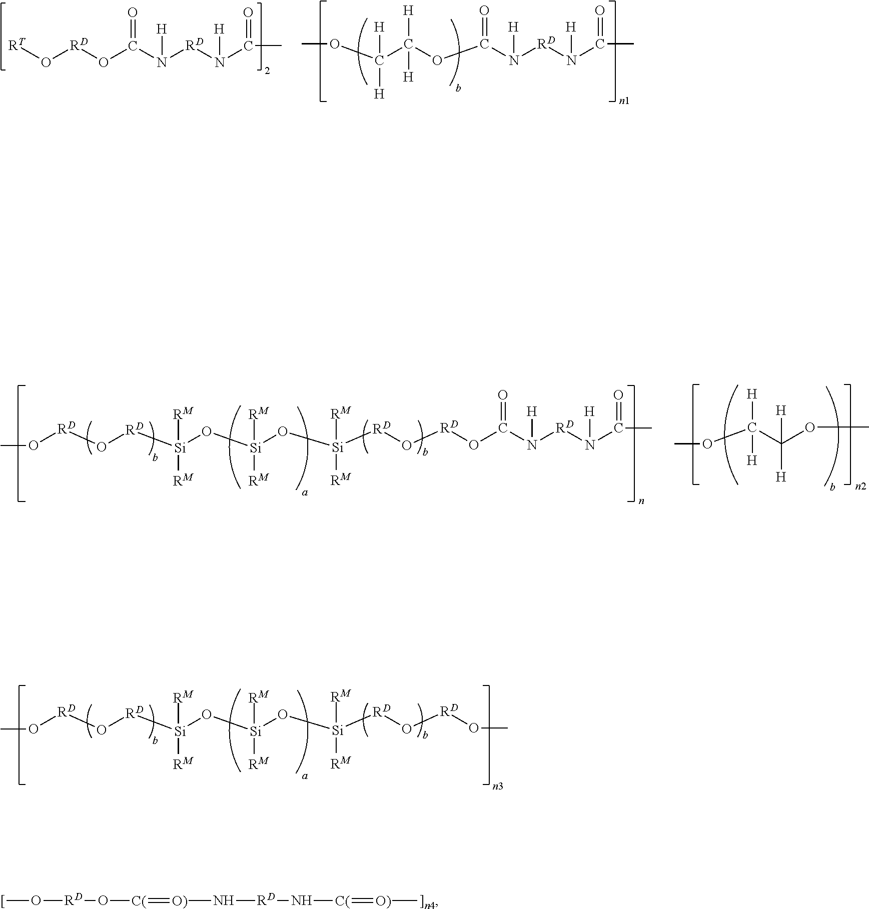

where R^{T}, R^{D}, R^{M}, subscripts a, b, and n are as described above, and subscript n1 is greater than or equal to 0, alternatively from 0 to 200,000, alternatively 0 to 20,000, alternatively 0 to 10,000, alternatively 0 to 5,000, alternatively 0 to 1,000, alternatively 0 to 100, alternatively 1 to 50. Subscripts n2 and n3 are each 0 or 1, and a quantity (n2+n3)=1. Subscript n4 is greater than or equal to 1.

Starting material (B) is an organic polyol. Suitable organic polyols are organic polymers containing two or more hydroxyl groups. The organic polyol for starting material (B) may be a polyether polyol, a polyester polyol, a polyacrylate polyol, a polycaprolactone polyol, a polyurethane polyol, a polycarbonate polyol, polybutadiene diol, other polymer polyols, or two or more of these organic polyols. Copolymer polyols of two or more types of polymers can also be used. Polyols with other modifications on the polymer structures, such as fluorination, can also be used. Suitable organic polyols alternatively may be an organic polymer diol. Such organic polymer diols include polyalkylene oxide diols e.g., polyethylene oxide diols, polypropylene oxide diols, and polybutylene oxide diols; or polycarbonate diols. Suitable organic polyols alternatively may be small molecule organic diols. Such small molecule organic diols include glycerol. The organic polyol may be added to tune the surface energy and/or hydrophilicity/mechanical properties of the copolymer composition. The amount added may be 0 to 95%, alternatively 0 to 75%, alternatively 0 to 50%, and alternatively 1 to 25%.

Starting material (C) can be prepared by reacting starting material (B), the organic polyol described above, with an isocyanate compound, which has an average of one or more isocyanate groups per molecule. Alternatively, the organic isocyanate compound may have an average of two or more isocyanate groups per molecule. The organic isocyanate compound may have formula: R—(N═C═O)ₚ where R is a hydrocarbon group or a halogenated hydrocarbon group and subscript p is an integer representing the number of isocyanate groups per molecule, and p is greater than or equal to 1. Alternatively subscript p is 2, 3, or 4; alternatively subscript p is 2 or 3; and alternatively, subscript p is 2. R is a divalent hydrocarbon group when subscript p is 2. R is a trivalent hydrocarbon group when subscript p is 3. R is a tetravalent hydrocarbon group when subscript p is 4.

The organic isocyanate compound is exemplified by monomeric isocyanates and polymeric isocyanates. Monomeric isocyanates include aromatic diisocyanates such as , meta-tetramethyl xylene diisocyanate (TMXDI), toluene diisocyanate (TDI), phenylene diisocyanate, xylene diisocyanate, 1,5-naphthalene diisocyanate, chlorophenylene 2,4-diisocyanate, bitoluene diisocyanate, dianisidine diisocyanate, toluidine diisocyanate and alkylated benzene diisocyanates; aliphatic and cycloaliphatic isocyanates such as hexamethylene diisocyanate (HDI), hydrogenated methylene diphenyl diisocyanate (HMDI), 1-isocyanato-3-isocyanatomethyl-3,5,5-trimethyl-cyclohexane (isophorone diisocyanate, IPDI), and nonanetriisocyanate (TTI), methylene-interrupted aromatic diisocyanates such as methylene-diphenyl-diisocyanate, especially the 4,4′-isomer (MDI) including alkylated analogs such as 3,3′-dimethyl-4,4′-diphenyl-methane diisocyanate; hydrogenated materials such as cyclohexylene diisocyanate, 4,4′-methylenedicyclohexyl diisocyanate; mixed aralkyl diisocyanates such as the tetramethylxylyl diisocyanates, 1,4-bis(1-isocyanato-1,1′-dimethylmethyl) benzene OCNC(CH₃)₂C₆H₄C(CH₃)₂NCO, and polymethylene isocyanates such as 1,4-tetramethylene diisocyanate, 1,5-pentamethylene diisocyanate, 1,6-hexamethylene diisocyanate (HDI), 1,7-heptamethylene diisocyanate, 2,2,4- and 2,4,4-trimethylhexamethylene diisocyanate, 1,10-decamethylene diisocyanate, and 2-methyl-1,5-pentamethylene diisocyanate; vinylisocyanate; and combinations thereof.

Polymeric organic isocyanates include dimerized isocyanates uretdiones or uretidinediones and carbodiimide, trimerized isocyanates isocyanurates, iminooxadiazine dione, uretonimine, and linear polymer α-Nylon; and derivatized isocyanates by reacting difuntional or multifunctional isocyanates with various compounds to form allophanate, or biuret compounds, or isocyanate functional urethane or other prepolymers. Some of the polyisocyanates are difunctional, i.e., having 2 isocyanate groups per molecule. Some have more than two isocyanate groups. An example is polymeric diphenylmethane diisocyanate, which is a mixture of molecules with two-, three-, and four- or more isocyanate groups, which may have an average functionality greater than two, commonly 2.7. Isocyanate functional compounds with isocyanate functionality greater than two may act as crosslinking sites. Commercially available isocyanate functional organic compounds are illustrated by Tolonate XIDT 70SB, an isophorone diisocyanate trimer (70% solids, 12.3 wt % NCO) sold by Rhodia (Cranbury, N.J.) and Desmodur N-100 polyisocyanate (available from Mobay Corp.).

The organic isocyanate compound can alternatively be a blocked isocyanate. The isocyanate group can be blocked by common blocking agents such as phenol, nonyl phenol, butanone oxime, caprolactam, and others. These blocked isocyanates can be release at a certain temperature to react with chain extenders and polyorganosiloxanes to construct an organic-siloxane copolymer. The blocking agent can react off/be released by heating to a certain temperature.

The reaction product (C) can be a low molecular weight compound, or a pre-polymer with low to medium molecular weight, or a high molecular weight polymer, depending on the isocyanate/OH reactive group molar ratio and the extent to which the reaction is carried out. The organic polyols can have a relatively large molecular weight and low glass transition temperature (Tg) so as to form a part of “soft segments” in the reaction product, or a low molecular weight so as to form the “hard segments” in the reaction product. Depending on the molar ratios of the polyols to isocyanates and the reaction, the reaction product (C) may have residual hydroxyl groups, or isocyanate groups, or both isocyanate group and hydroxyl groups, or no residual reactive groups.

The method of making reaction product (C) from the starting materials (i.e., organic polyol and polyisocyanate) are known, and any conventional method to make a polyurethane polymer can be employed. Such methods can be found in U.S. Pat. Nos. 3,384,623; 5,200,491; and 5,621,024.

The personal care composition described above includes copolymer (A), which may be useful as a film forming agent in the personal care composition. The personal care composition may comprise one copolymer (A) or a combination comprising two or more different species of copolymer (A). The personal care composition may optionally further comprise one or both of starting material (B) the organic polyol; and starting material (C) the reaction product of an organic polyisocyanate and an organic polyol. The personal care composition may comprise (A) and (B). Alternatively, the personal composition may comprise (A) and (C). Alternatively, the personal care composition may comprise (A), (B), and (C).

The method to make copolymer (A) is similar to the method to make the reaction product (C). The methods described in the references cited above may be used but varying the starting materials to those described herein.

The copolymer described above as starting material (A) may be prepared by a method comprising
1) reacting starting materials comprising:
a) an isocyanate compound,
b) a polyorganosiloxane, and
c) a chain extender.

In one embodiment, all starting materials may be combined and reacted concurrently. Alternatively, starting materials comprising a) the isocyanate compound and b) the polyorganosiloxane may be reacted to form a prepolymer, and thereafter the prepolymer may be reacted with a starting material comprising c) the chain extender, and optionally with an additional amount of a) the isocyanate compound to form the copolymer. Alternatively, starting materials comprising a) the isocyanate compound and c) the chain extender may be reacted to form an intermediate, and thereafter the intermediate may be reacted with a starting material comprising b) the polyorganosiloxane and optionally an additional amount of a) the isocyanate compound to form the copolymer.

In all these embodiments, wherever a polyorganosiloxane is reacted, a mixture of polyorganosiloxane and an organic polyol can be used in place of the polyorganosiloxane. Alternatively, the method may comprise: i) reacting a) the isocyanate compound with b) the polyorganosiloxane and d) an organic polyol to form a prepolymer, and thereafter ii) reacting the prepolymer with c) the chain extender, and optionally an additional amount of a) the isocyanate compound.

In each embodiment of the method described above, starting material b) the polyorganosiloxane may be b1) a carbinol-functional polyorganosiloxane, b2) an amine-functional polyorganosiloxane, or a mixture of both b1) and b2).

In the method described above, a) the isocyanate compound has an average of one or more isocyanate groups per molecule. Alternatively, the isocyanate compound may have an average of two or more isocyanate groups per molecule. The isocyanate compound may have formula: R—(N═C═O)ₚ, where R is a polyvalent hydrocarbon group or a polyvalent halogenated hydrocarbon group and subscript p is an integer representing the number of isocyanate groups per molecule. Subscript p is greater than or equal to 1. Alternatively, subscript p is 2, 3, or 4; alternatively subscript p is 2 or 3; and alternatively, subscript p is 2. R is a divalent hydrocarbon group when subscript p is 2. R is a trivalent hydrocarbon group when subscript p is 3. R is a tetravalent hydrocarbon group when subscript p is 4.

The isocyanate compound is exemplified by monomeric isocyanates and polymeric isocyanates. Monomeric isocyanates include aromatic diisocyanates such as meta-tetramethyl xylene diisocyanate (TMXDI), toluene diisocyanate (TDI), phenylene diisocyanate, xylene diisocyanate, 1,5-naphthalene diisocyanate, chlorophenylene 2,4-diisocyanate, bitoluene diisocyanate, dianisidine diisocyanate, toluidine diisocyanate and alkylated benzene diisocyanates; aliphatic and cycloaliphatic isocyanates such as hexamethylene diisocyanate (HDI), hydrogenated methylene diphenyl diisocyanate (HMDI), 1-isocyanato-3-isocyanatomethyl-3,5,5-trimethyl-cyclohexane (isophorone diisocyanate, IPDI), and nonanetriisocyanate (TTI), methylene-interrupted aromatic diisocyanates such as methylene-diphenyl-diisocyanate, especially the 4,4′-isomer (MDI) including alkylated analogs such as 3,3′-dimethyl-4,4′-diphenyl-methane diisocyanate; hydrogenated materials such as cyclohexylene diisocyanate, 4,4′-methylenedicyclohexyl diisocyanate; mixed aralkyl diisocyanates such as the tetramethylxylyl diisocyanates, 1,4-bis(1-isocyanato-1,1′-dimethylmethyl) benzene OCNC(CH₃)₂C₆H₄C(CH₃)₂NCO, and polymethylene isocyanates such as 1,4-tetramethylene diisocyanate, 1,5-pentamethylene diisocyanate, 1,6-hexamethylene diisocyanate (HDI), 1,7-heptamethylene diisocyanate, 2,2,4- and 2,4,4-trimethylhexamethylene diisocyanate, 1,10-decamethylene diisocyanate, and 2-methyl-1,5-pentamethylene diisocyanate, vinylisocyanate; and combinations thereof.

Polymeric isocyanates include dimerized isocyanates, uretdiones or uretidinediones, and carbodiimide, trimerized isocyanates, isocyanurates, iminooxadiazine dione, uretonimine, and linear polymer α-Nylon; and derivatized isocyanates by reacting difunctional or multifunctional isocyanates with various compounds to form allophanate, or biuret compounds, or isocyanate functional urethane or other prepolymers. Some of the polyisocyanates are difunctional, i.e., having 2 isocyanate groups per molecule. Some have more than two isocyanate groups. An example is polymeric diphenylmethane diisocyanate, which is a mixture of molecules with two-, three-, and four- or more isocyanate groups, which may have an average functionality greater than two, commonly 2.7. Isocyanate functional compounds with isocyanate functionality greater than two may act as crosslinking sites. Commercially available isocyanate functional organic compounds are illustrated by Tolonate XIDT 70SB, an isophorone diisocyanate trimer (70% solids, 12.3 wt % NCO) sold by Rhodia (Cranbury, N.J.) and Desmodur N-100 polyisocyanate (available from Mobay Corp.).

Alternatively, a) the isocyanate compound may comprise a blocked isocyanate. The isocyanate group can be blocked by common blocking agents such as phenol, nonyl phenol, butanone oxime, caprolactam, and others. These blocked isocyanates can be released by any conventional means such as heating at a temperature above room temperature to react with chain extenders and polyorganosiloxanes to construct the polyurethane-polyorganosiloxane copolymer.

In the method described above, b1) the carbinol-functional polyorganosiloxane comprises units of formulae:

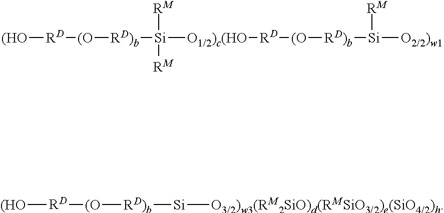

In this unit formula, each R^{M}, R^{D}, subscript b, subscript c, subscript w1, subscript w3, subscript d, subscript e, and subscript h are as described above. Examples of carbinol-functional polyorganosiloxanes are disclosed in WO2008/088491, U.S. Pat. Nos. 6,528,121, and 7,452,956. The carbinol groups can be terminal or pendent. Alternatively, the carbinol groups may be terminal.

Alternatively, b1) the carbinol-functional polyorganosiloxane may comprise an α,ω-difunctional polydiorganosiloxane of formula (II):

$${R}^{C} {R}_{2}^{M} Si - {R}^{D X} - {( {R}_{2}^{M} SiO )}_{l} - ( {R}_{2}^{M} ) {SiR}^{DX} - {SiR}_{2}^{M} {R}^{C} ,$$

where, each R^{C} is independently a carbinol functional group of formula HO—R^{D}—(ORD)_{b}- where subscript b, R^{M} and R^{D} are as described above, each R^{DX} is independently selected from O or a divalent hydrocarbon group described above as R^{D}, and subscript r represents the degree of polymerization of the carbinol-functional polyorganosiloxane of formula (II). Subscript r>0. Alternatively, subscript r may be 1 to 1,000,000, alternatively 50 to 1,000, and alternatively 200 to 700. Alternatively, subscript r is 0 to 200,000, alternatively 0 to 200,000, alternatively 0 to 100,000, alternatively 0 to 50,000, alternatively 0 to 10,000, alternatively 0 to 5,000, alternatively 0 to 1,000, alternatively 1 to 1,000, alternatively 1 to 500, alternatively 1 to 200, and alternatively 5 to 150. Alternatively, each R^{DX} is O.

The amine functional polyorganosiloxane comprises units of formulae:

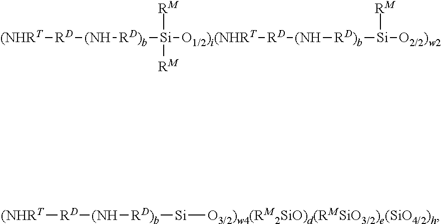

where R^{M} and R^{D}, and subscripts b, d, e, h, and i are as described above. The amine groups can be terminal or pendent. Alternatively, the amine groups can be terminal.

An exemplary amine terminated polyorganosiloxane comprises a terminal unit of formula

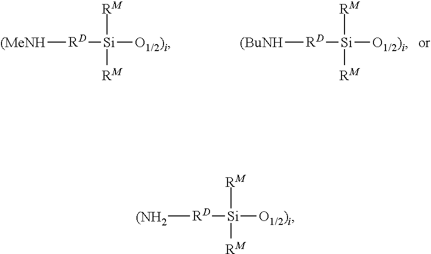

where Me represents a methyl group and Bu represents a butyl group; and further comprises units comprising one or more of

$${( {R}_{2}^{M} {SiO}_{2 / 2} )}_{d} {( {R}^{M} {SiO}_{3 / 2} )}_{e} {( {SiO}_{4 / 2} )}_{h} ,$$

where R^{M}, R^{D}, and subscripts i, d, e, and h are as described above.

The chain extender may be a dialcohol, of formula HO—R^{D}—OH, where R^{D} is as defined above. Suitable dialcohols include 1,3-butanediol; 1,4-butanediol; 1,6-hexanediol, 1,10-decanediol; 1,6-hexamethylenediol; 2,2-dimethyl-1,3-propanediol; 1,4-cyclohexanedimethylol; 1,1′-isopropylidine-bis-(p-phenylene-oxy)-di-2-ethanol; poly(tetrmethylene ether) glycol; and ethylene glycol. Alternatively, the chain extender may be a diamine containing 2 to 20 carbon atoms e.g., 1,2-diaminoethane; 1,4-diaminobutane; 1,2-propanediamine; hexamethylenediamine; diethylene diamine; 5-amino-1-(aminomethyl)-1,3,3-trimethylcyclohexane; 4,4′-methylene bis(cyclohexylamine); and ethanol amine. Alternatively, the chain extender may be a dithiol, a dicarboxylic acid, or a diepoxide. Suitable chain extenders are disclosed, for example, in U.S. Pat. Nos. 4,840,796 and 5,756,572.

A solvent may be added during the method to prepare a copolymer described herein. Any organic compoound that will dissolve the copolymer and that is relatively unreactive towards isocyanate, and amine and/or carbinol compounds is suitable as a solvent. Examples include aliphatic hydrocarbons, aromatic hydrocarbons, esters, ethers, ketones, and amides. Exemplary solvents include methyl ethyl ketone, ethyl acetate, butyl acetate, or tetrahydrofuran.

The amount of solvent to be used depends on the properties of the copolymer including structure, molecular weight, and the particular method of copolymer preparation, and can be 0 to 99%. Generally for higher molecular weight copolymers especially when a high torque mixing mechanim will not be used, solvent may be added to reduce the viscosity and make the system easier to handle during performance of the method to make the copolymer. If the molecular weight is relatively low and/or high torque mixing equipment such as a twin screw extruder is used, no solvent needs to be used. When solvent is used, the amount may be 0 to 99%, alternatively 0 to 80%, alternatively 1% to 60%, and alternatively 5% to 50%, based on the combined weights of all starting materials used.

The amounts of starting materials a), b), c), and when present, d) and/or e), can vary widely, according to the polyorganosiloxane structure and molecular weight desired, to arrive at the copolymer described by the formula herein. The molar ratio of isocyanate groups of starting material a) to the active hydrogen of carbinol or amine groups on the polysiloxane selected for starting material b) can be 0.1 to 100, alternatively 0.1 to 50, alternatively 0.1 to 10, alternatively 0.1 to 2, alternatively 0.1 to 1.5, alternatively 0.1 to 1.25, alternatively 0.1 to 1.1, alternatively 0.1 to 1.05, alternatively 0.1 to 1.01, alternatively 0.1 to 1, alternatively 0.1 to 0.9, alternatively 0.1 to 0.5, alternatively 0.5 to 50, alternatively 0.5 to 10, alternatively 0.5 to 2, alternatively 0.5 to 1.5, alternatively 0.5 to 1.25, alternatively 0.5 to 1.1, alternatively 0.5 to 1.05, alternatively 0.5 to 1.01, alternatively 0.5 to 1, alternatively 0.5 to 0.9, and alternatively 0.4 to 0.7.

The molar ratio between the isocyanate groups to the active hydrogen on the hydroxyl or amine groups or other reactive groups on the chain extender can be 1.001 to 1,000,000, alternatively 1.001 to 500,000, alternatively 1.001 to 200,000, alternatively 1.001 to 100,000, alternatively 1.001 to 50,000, alternatively 1.001 to 10,000, alternatively 1.001 to 5,000, alternatively 1.001 to 1,000, alternatively 1.001 to 500, alternatively 1.001 to 100, alternatively 1.001 to 50, alternatively 1.001 to 20, alternatively 1.001 to 10, alternatively 1.001 to 5, alternatively 1.001 to 4, alternatively 1.001 to 3, alternatively 1.001 to 2, alternatively 1.001 to 1.5, alternatively 1.001 to 1.3, alternatively 1.001 to 1.2, alternatively 1.01 to 20, alternatively 1.01 to 10, alternatively 1.01 to 5, alternatively 1.01 to 4, alternatively 1.01 to 3, alternatively 1.01 to 2, alternatively 1.01 to 1.5, alternatively 1.01 to 1.3, and alternatively 1.01 to 1.2.

Reacting the starting materials comprising a), b), and c) described above may be catalyzed by starting material e) a catalyst. Suitable catalysts include tertiary amines and metal salts, for example, the salts of tin. Tin compounds are useful as catalysts herein include those where the oxidation state of the tin is either +4 or +2, i.e., tin (IV) compounds or tin (II) compounds. Examples of tin (IV) compounds include stannic salts such as dibutyl tin dilaurate, dimethyl tin dilaurate, di-(n-butyl)tin bis-ketonate, dibutyl tin diacetate, dibutyl tin maleate, dibutyl tin diacetylacetonate, dibutyl tin dimethoxide, carbomethoxyphenyl tin tris-uberate, dibutyl tin dioctanoate, dibutyl tin diformate, isobutyl tin triceroate, dimethyl tin dibutyrate, dimethyl tin di-neodecanoate, dibutyl tin di-neodecanoate, triethyl tin tartrate, dibutyl tin dibenzoate, butyltintri-2-ethylhexanoate, dioctyl tin diacetate, tin octylate, tin oleate, tin butyrate, tin naphthenate, dimethyl tin dichloride, a combination thereof, and/or a partial hydrolysis product thereof. Tin (IV) compounds are known in the art and are commercially available, such as Metatin® 740 and Fascat® 4202 from Acima Specialty Chemicals of Switzerland, Europe, which is a business unit of The Dow Chemical Company. Examples of tin (II) compounds include tin (II) salts of organic carboxylic acids such as tin (II) diacetate, tin (II) dioctanoate, tin (II) diethylhexanoate, tin (II) dilaurate, stannous salts of carboxylic acids such as stannous octoate, stannous oleate, stannous acetate, stannous laurate, stannous stearate, stannous naphthanate, stannous hexanoate, stannous succinate, stannous caprylate, and a combination thereof. Other metal salts are also suitable catalysts for this reaction. Examples include zinc salts such as zinc acetate and zinc naphthenate. Salts of lead, bismuth, cobalt, iron, antimony, or sodium, such as lead octoate, bismuth nitrate, and sodium acetate can also catalyze this reaction. In certain occasions organomercuric compounds can also be used. Optionally co-catalysts can also be used along with a catalyst described above. Alternatively, a combination of two or more catalysts can be used, e.g., to provide either faster reaction than achievable with a single catalyst, or a better balanced reaction initiation time and finish time.

An organic polyol may optionally be combined with b) the polyorganosiloxane to make the copolymer (A) and/or copolymer (B) described above. Suitable organic polyols are organic polymers containing two or more hydroxyl groups. The organic polyol for starting material f) may be a polyether polyol, a polyester polyol, a polyacrylate polyol, a polycaprolactone polyol, a polyurethane polyol, a polycarbonate polyol, polybutadiene diol, other polymer polyols, or two or more of these organic polyols. Copolymer polyols of two or more types of polyols can also be used. Polymeric polyols with other modifications on the polymer structures, such as fluorination, can also be used. Suitable organic polyols alternatively may be organic diols. Suitable organic diols include polyalkylene oxide diols e.g., polyethylene oxide diols, polypropylene oxide diols, and polybutylene oxide diols; or polycarbonate diols. Other organic diols include glycerol. The organic polyol may be added to tune the surface energy and/or hydrophilicity/mechanical properties of the copolymer being prepared. The amount added may be 0 to 95%, alternatively 0 to 75%, alternatively 0 to 50%, and alternatively 1 to 25% based on combined weights of all starting materials used to make the copolymer.

The copolymer (A) and/or (B) described above may optionally be reacted with an enblocker to convert any residual isocyanate groups, hydroxyl groups, or amine groups to another type of reactive or non-reactive group. Suitable endblockers include but are not limited to alcohols such ethanol, propanol, butanol, carboxylic acids such as acetic acids, and alcohols and carboxylic acids containing aliphatic unsaturation. Thio-alcohols, hydroxylamines, glycol, amino acids, and amino sugars are also suitable as endblockers.

The method described above may be performed with or without heating. The temperature for the reaction depends on the selection of starting materials a), b), and c) and whether any of d), e), f), and/or g) is present, however, the temperature may range from −20° C. to 150° C.; alternatively 0° C. to 100° C., and alternatively 20° C. to 60° C. at pressure of 1 atmosphere. Pressure under which the method is performed is not critical.

The method described above may be peformed in batch, semi-batch, semi-continuous, or continuous mode in any convenient equipment. When preparing higher molecular weight copolymers (e.g., when higher molecular weight starting materials are used), the method may be performed in an extruder, such as a twin screw extruder. The copolymer described above may be prepared using the equipment and method as described in U.S. Pat. No. 5,756,572, except using the strating materials described above.

Copolymer (A), described above, optionally further comprising starting material (B) and/or starting material (C), described above, is useful as a film forming agent. Copolymer (A) is useful in personal care compositions. The personal care compositions are suitable for application to various substrates including skin or hair, e.g., human skin or human hair. The copolymer may act as a film forming agent in such personal care compositions. In addition to the copolymer as described above, the personal care composition further comprises a carrier that permits application to the substrate.

Suitable carriers for personal care applications, such as skin care, include nonaqueous media capable of evaporating on contact with the skin in less than one hour, at room temperature and atmospheric pressure, e.g., isoparaffins such as isododecane and silicone oils such as caprylyl methicone.

Alternatively, the carrier may comprise a surfactant and water, e.g., the composition may form an emulsion comprising (1) copolymer (A) described above, (2) a surfactant, and (3) water. As used herein, “emulsion” is meant to encompass water continuous emulsions (for example an oil in water type emulsion (o/w), or a silicone in water emulsion (s/w)), oil or silicone continuous emulsions (water in oil emulsions (w/o) or water in silicone emulsions (w/s)), or multiple emulsions (water/oil/water, oil/water/oil types, water/silicone/water, or silicone/water/silicone). Copolymer (A) may be added to any type of emulsion by common mixing techniques. The addition of copolymer (A) may occur either during the preparation of the emulsion, or subsequently post added to a pre-formed emulsion. There are no special requirements or conditions needed to effect the mixing of copolymer (A) and the emulsion. Mixing techniques can be simple stirring, homogenizing, sonolating, and other mixing techniques known in the art to effect the formation of emulsions. The mixing can be conducted in a batch, semi-continuous, or continuous process.

The amount of copolymer (A) added to the emulsion can vary and is not limited, however the amounts may range from a copolymer /emulsion weight ratio of 0.1/99 to 99/0.1, alternatively 1/99 to 99/1.

The emulsions used may be w/o, w/s, or multiple phase emulsions using silicone emulsifiers. In one embodiment, the water-in-silicone emulsifier in such formulation is non-ionic and is selected from polyoxyalkylene-substituted silicones, silicone alkanolamides, silicone esters and silicone glycosides. Silicone-based surfactants may be used to form such emulsions and have been described, for example, in U.S. Pat. No. 4,122,029 to Gee et al., U.S. Pat. No. 5,387,417 to Rentsch, and U.S. Pat. No. 5,811,487 to Schulz et al.

Alternatively, the emulsion containing the copolymer may contain anionic surfactants, cationic surfactants, amphoteric surfactants, and nonionic surfactants. The anionic surfactants include (i) sulfonic acids and their salt derivatives, including alkyl, aralkyl, alkylnaphthalene, alkyldiphenyl ether sulfonic acids, and their salts, having at least 6 carbon atoms in the alkyl substituent, such as dodecylbenzene sulfonic acid, and its sodium salt or its amine salt; (ii) alkyl sulfates having at least 6 carbon atoms in the alkyl substituent, such as sodium lauryl sulfate; (iii) the sulfate esters of polyoxyethylene monoalkyl ethers; (iv) long chain carboxylic acid surfactants and their salts, such as lauric acid, steric acid, oleic acid, and their alkali metal and amine salts. Some other examples of anionic surfactants are alkali metal sulfosuccinates; sulfonated glyceryl esters of fatty acids such as sulfonated monoglycerides of coconut oil acids; salts of sulfonated monovalent alcohol esters such as sodium oleyl isothionate; amides of amino sulfonic acids such as the sodium salt of oleyl methyl tauride; sulfonated products of fatty acid nitriles such as palmitonitrile sulfonate; sulfonated aromatic hydrocarbons such as sodium alpha-naphthalene monosulfonate; condensation products of naphthalene sulfonic acids with formaldehyde; sodium octahydro anthracene sulfonate; alkali metal alkyl sulfates; ether sulfates having alkyl groups of eight or more carbon atoms such as sodium lauryl ether sulfate; and alkylaryl sulfonates having one or more alkyl groups of eight or more carbon atoms such as neutral salts of hexadecylbenzene sulfonic acid and C₂₀ alkylbenzene sulfonic acid.

Commercial anionic surfactants which can be used include the sodium salt of dodecylbenzene sulfonic acid sold under the trademark SIPONATE® DS-10 by Alcolac Inc., Baltimore, Maryland; sodium n-hexadecyl diphenyloxide disulfonate sold under the trademark DOWFAX® 8390 by The Dow Chemical Company, Midland, Michigan; the sodium salt of a secondary alkane sulfonate sold under the trademark HOSTAPUR® SAS 60 by Clariant Corporation, Charlotte, N.C.; N-acyl taurates such as sodium N-lauroyl methyl taurate sold under the trademark NIKKOL LMT® by Nikko Chemicals Company, Ltd., Tokyo, Japan; and linear alkyl benzene sulfonic acids sold under the trademark BIO-SOFT® S-100 by the Stepan Company, Northfield, Ill. Compositions of the latter type such as dodecylbenzene sulfonic acid, although a catalyst as noted above, can also function as the anionic surfactant when neutralized. Other suitable surfactants include sodium alkyl sulfonate such as HOSTAPUR® SAS-30. In one embodiment, the emulsifier is triethanolamine dodecylbenzene sulfonate, such as BIO-SOFT® N 300.

Cationic surfactants useful herein include compounds containing quaternary ammonium hydrophilic moieties in the molecule which are positively charged, such as quaternary ammonium salts represented by R⁸R⁹R¹⁰R¹¹N⁺X⁻ where R⁸ to R¹¹ are alkyl groups containing 1-30 carbon atoms, or alkyl groups derived from tallow, coconut oil, or soy; and X is a halogen, e.g., chlorine or bromine. Alternatively, the quaternary ammonium compounds may be alkyl trimethylammonium and dialkyldimethylammonium halides, or acetates, or hydroxides, having at least 8 carbon atoms in each alkyl substituent. Dialkyl dimethyl ammonium salts can be used and are represented by R¹²R¹³N⁺(CH₃)₂X⁻ where R¹² and R¹³ are alkyl groups containing 12-30 carbon atoms or alkyl groups derived from tallow, coconut oil, or soy; and X is halogen. Monoalkyl trimethyl ammonium salts can be used and are represented by R¹⁴N⁺(CH₃)₃X⁻ where R¹⁴ is an alkyl group containing 12-30 carbon atoms or an alkyl group derived from tallow, coconut oil, or soy; and X is halogen, acetate, or hydroxide.

Representative quaternary ammonium halide salts are dodecyltrimethyl ammonium chloride/lauryltrimethyl ammonium chloride (LTAC), cetyltrimethyl ammonium chloride (CTAC), didodecyldimethyl ammonium bromide, dihexadecyldimethyl ammonium chloride, dihexadecyldimethyl ammonium bromide, dioctadecyldimethyl ammonium chloride, dieicosyldimethyl ammonium chloride, didocosyldimethyl ammonium chloride, dicoconutdimethyl ammonium chloride, ditallowdimethyl ammonium chloride, and ditallowdimethyl ammonium bromide. These quaternary ammonium salts are commercially available under trademarks such as ADOGEN®, ARQUAD®, TOMAH®, and VARIQUAT®.

Other suitable cationic surfactants which can be used include (i) fatty acid amines and amides and their salts and derivatives, such as aliphatic fatty amines and their derivatives. Such cationic surfactants that are commercially available include compositions sold under the names Arquad T27 W, Arquad 16-29, by Akzo Nobel Chemicals Inc., Chicago, Ill.; and Ammonyx Cetac-30 by the Stepan Company, Northfield, Ill.

Suitable amphoteric surfactants include; betaines such as cocamidopropylbetaine, sultaines such as cocamidopropylhydroxysultaine, lecithin and hydrogenated lecithin, In one embodiment, the emulsifier is a combination of an anionic and nonionic surfactant. In a further embodiment, the anionic surfactant in the combination is an alkyl sulfonate or a dodecylbenzene sulfonate. In a further embodiment, the nonionic emulsifier is an alkyl-oxo alcohol polyglycol ether or an alkyl polyethylene glycol ether.

Some suitable nonionic surfactants which can be used include polyoxyethylene alkyl ethers, polyoxyethylene alkyl phenyl ethers, alkylglucosides, polyoxyethylene fatty acid esters, sorbitan fatty acid esters, and polyoxyethylene sorbitan fatty acid esters. Nonionic surfactants which are commercially available include compositions such as (i) 2,6,8-trimethyl-4-nonyl polyoxyethylene ether sold under the names Tergitol TMN-6 and Tergitol TMN-10; (ii) the C11-15 secondary alkyl polyoxyethylene ethers sold under the names Tergitol 15-S-7, Tergitol 15-S-9, Tergitol 15-S-15, Tergitol 15-S-30, and Tergitol 15-S-40, by the Dow Chemical Company, Midland, Mich.; octylphenyl polyoxyethylene (40) ether sold under the name Triton X405 by the Dow Chemical Company, Midland, Mich.; (iii) nonylphenyl polyoxyethylene (10) ether sold under the name Makon 10 by the Stepan Company, Northfield, Ill.; (iv) ethoxylated alcohols sold under the name Trycol 5953 by Henkel Corp./Emery Group, Cincinnati, Ohio; (v) ethoxylated alcohols sold under the name Brij L23 and Brij L4 by Croda Inc. Edison, N.J., (vi) alkyl-oxo alcohol polyglycol ethers such as ®GENAPOL UD 050, and Genapol UD110, (vii) alkyl polyethylene glycol ether based on C10-Guerbet alcohol and ethylene oxide such as LUTENSOL® XP 79.

Suitable nonionic surfactants also include poly(oxyethylene)-poly(oxypropylene)-poly(oxyethylene) tri-block copolymers. Poly(oxyethylene)-poly(oxypropylene)-poly(oxyethylene) tri-block copolymers are also commonly known as Poloxamers. They are nonionic triblock copolymers composed of a central hydrophobic chain of polyoxypropylene (poly(propylene oxide)) flanked by two hydrophilic chains of polyoxyethylene (poly(ethylene oxide)). Poly(oxyethylene)-poly(oxypropylene)-poly(oxyethylene) tri-block copolymers are commercially available from BASF (Florham Park, N.J.) and are sold under the tradename PLURONIC®, such as Pluronic L61, L62, L64, L81, P84.

The nonionic surfactant may also be a silicone polyether (SPE). The silicone polyether as an emulsifier may have a rake type structure wherein the polyoxyethylene or polyoxyethylene-polyoxypropylene copolymeric units are grafted onto the siloxane backbone, or the SPE can have an ABA block copolymeric structure wherein A represents the polyether portion and B the siloxane portion of an ABA structure. Suitable silicone polyethers include Dow Corning® 5329 from Dow Corning Corporation of Midland, Mich. USA.

Other useful commercial nonionic surfactants are nonylphenoxy polyethoxy ethanol (10EO) sold under the trademark MAKON® 10 by Stepan Company, Northfield, Ill.; polyoxyethylene 23 lauryl ether (Laureth-23) sold commercially under the trademark BRIJ® 35L by ICI Surfactants, Wilmington, Del.; and RENEX® 30, a polyoxyethylene ether alcohol sold by ICI Surfactants, Wilmington, Del.

Protective colloids, i.e., colloidal stabilizers, may be used, if desired, to enhance stability or to provide a specific rheological characteristic to the emulsion. As used herein, the terms “protective colloid” and/or “colloidal stabilizer” mean a nonionic molecule that is an effective agent for protecting charged colloidal particles in an aqueous media against flocculation. These compositions typically have a weight average molecular weight ranging from 1,000-300,000 and are typically more hydrophilic than the composition of the first emulsion polymer, as measured by weight-averaged solubility parameters. Colloidal stabilizers which can be used include hydroxyethyl cellulose having a weight average molecular weight between 50,000-150,000; N-vinyl pyrrolidone; polyvinyl alcohol having a weight average molecular weight between 10,000-200,000; partially acetylated polyvinyl alcohol; carboxymethyl cellulose; gums such as gum arabic; starches; proteins; and mixtures thereof. Preferred colloidal stabilizers are hydroxethyl cellulose and polyvinyl alcohol.

Since emulsions are susceptible to microbiological contamination a preservative can be added. Representative preservatives, which can be used include phenoxyethanol and ethylhexylglycerin; formaldehyde; 1,3-dimethylol-5,5-dimethyl hydantoin, e.g., DMDM Hydantoin; 5-bromo-5-nitro-1,3-dioxane; methyl or propyl paraben; sorbic acid; imidazolidinyl urea; and KATHON® CG (5-chloro-2-methyl-4-isothiazolin-3-one); caprylyl glycol; phenoxyethanol; benzyl alcohol; and/or benzoic acid.

The emulsions may contain concentration of copolymer (A) of 1% to 70% based on the weight of the total emulsion, alternatively 2% to 60%. While emulsions containing less than 1% copolymer (A) content can be made, such emulsions may be less valuable. The surfactant may be present at 0.05% to 30% based on the weight of the total emulsion, alternatively 0.1% to 20%. Water and optional ingredients constitute the balance of the emulsion to 100%.

The composition comprising copolymer (A) may comprise 1% to 70%, alternatively 2% to 65%, alternatively 5% to 60%, and alternatively 20% to 50% of the copolymer (A), based on the weight of all ingredients in the composition. The carrier may be present in an amount sufficient to form a solution, dispersion or emulsion of copolymer (A). In one embodiment, the balance of the composition to 100% may be the carrier. Alternatively, the composition may further comprise one or more optional ingredients in addition to copolymer (A) and the carrier. The selection of additional ingredients depends on the end use of the composition. The composition comprising copolymer (A) and a carrier, as described above, may be used to prepare a personal care composition, e.g., a hair care and/or a skin care composition.

Non-limiting examples of additional ingredients which may be formulated into the personal care compositions in addition to copolymer (A) described above include: silicones (e.g., fluids, gums, resins, elastomers, surfactants, and/or alkylmethylsilicones, and/or silicone carbinol fluids), anti-oxidants, cleansing agents, colorants (e.g., pigments and/or dyes), conditioning agents, deposition agents, electrolytes, emollients, exfoliating agents, foam boosters, fragrances, humectants, occlusive agents, pediculicides, pH control agents, pigments, preservatives, biocides, solvents (other than the carrier), stabilizers, sun-screening agents, suspending agents, tanning agents, other surfactants (e.g., other than the surfactant used when the composition is an emulsion), thickeners, vitamins, botanicals, fragrances, waxes, rheology-modifying agents, anti-dandruff, anti-acne, anti-carie, anti-perspirant/deodorant actives, pharmaceutical ingredients, and wound healing-promotion agents.

The personal care compositions may be functional with respect to the portion of the body to which they are applied, cosmetic, therapeutic, or some combination thereof. Conventional examples of such personal care compositions include, but are not limited to: antiperspirants and/or deodorants; wound management, wound protection, and/or wound care compositions; skin barriers; liquid bandages; scar and/or stretch mark treatments; skin care creams; skin care lotions; moisturizers; facial treatments such as acne or wrinkle removers; personal and/or facial cleansers; bath oils; perfumes; colognes; sachets; sunscreens; pre-shave and/or after-shave lotions; shaving soaps and/or shaving lathers; hair shampoos; hair conditioners (either leave in or rinse off); hair colorants; hair relaxants; hair styling aids such as sprays, fixatives, mousses, gels, permanents, depilatories, and/or cuticle coats; make-ups; color cosmetics; foundations; concealers; blushes; lipsticks; eyeliners; mascara; oil removers; color cosmetic removers; powders; and/or medicament creams, pastes or sprays including antiacne, dental hygienic, antibiotic, healing promotive, and/or nutritive, which may be preventative and/or therapeutic. The siloxane-urethane-urea copolymer may be used as a film forming agent in any of such personal care compositions. In general the personal care compositions may be formulated with a carrier that permits application in any conventional form, including but not limited to liquids, rinses, lotions, creams, pastes, gels, foams, mousses, ointments, sprays, aerosols, soaps, sticks, soft solids, solid gels, and gels. Generally, such personal care compositions can be prepared at room temperature if no solid materials at room temperature is present in the composition, using simple propeller mixers, Brookfield counter-rotating mixers, or homogenizing mixers. No special equipment or processing conditions are typically required. Depending on the type of form made, the method of preparation will be different, but conventional methods may be used.

The personal care compositions according to this invention can be used by standard methods, such as applying them to the human body, e.g., skin or hair, using applicators, brushes, applying by hand, pouring them and/or possibly rubbing or massaging the composition onto or into the body. Removal methods, for example for color cosmetics are also well known standard methods, including washing, wiping, peeling and the like. For use on the skin, the personal care compositions according to the present invention may be used in a conventional manner for example for conditioning the skin. An effective amount of the composition for the purpose is applied to the skin. Such effective amounts generally range from 1 mg/cm² to 3 mg/cm². Application to the skin typically includes working the composition into the skin. This method for applying to the skin comprises the steps of contacting the skin with the composition in an effective amount and then rubbing the composition into the skin. These steps can be repeated as many times as desired to achieve the desired benefit.

The use of the personal care compositions according to the invention on hair may use a conventional manner for conditioning hair. An effective amount of the composition for cleaning and/or conditioning hair is applied to the hair. Such effective amounts generally range from 0.5 g to 50 g, alternatively from 1 g to 20 g. Application to the hair typically includes working the composition through the hair such that most or all of the hair is contacted with the product. This method for cleaning and/or conditioning the hair comprises the steps of applying an effective amount of the hair care product to the hair, and then working the composition through the hair. These steps can be repeated as many times as desired to achieve the desired conditioning benefit.

Exemplary skin care compositions that can be made with the composition comprising the siloxane-urethane-urea copolymer and the carrier, described above include, a foundation composition comprising: (1) the copolymer described above, (2) water, (3) a surfactant, (4) a thickener (e.g., sodium chloride), (5) an emollient (e.g., glycerin), (6) a preservative (e.g., phenoxyethanol), (7) a pigment, and (8) a silicone (e.g., a polydimethylsiloxane and/or an alkylmethylsiloxane).

Alternatively, the skin care composition comprising the siloxane-urethane-urea copolymer and the carrier, described above, may be a lipstick composition comprising: (1) the siloxane-urethane-urea copolymer described above, (2) a solvent (e.g. Isododecane and/or cyclomethicone), (3) an emollients (e.g., a vegetable oil, a plant oil, a silicone, and/or an ester), (4) a wax (e.g., polyethylene, ceresin, ozokerite, synthetic, paraffin, alkyl silicones, and/or beeswax), (5) a fixative (e.g., a silicone resins such as MQ or TPr silicone resin and/or a silicone copolymer other than the polyurethane-polyorganosiloxane copolymer described above), (6) a colorant (e.g., D&Cs Red #6, FD&C Yellow #5,6 Al Lake, Iron Oxides, TiO₂, ZnO, and/or Pearls), (7) an active (e.g., tocopherol acetate, sodium hyaluronate, amino acids, panthenol, and/or ascorbyl palmitate), (8) a fillers (e.g. mica, silica, boron nitride, starch, and/or acrylate copolymer), (9) an antioxidants and/or preservative (e.g., phenoxyethanol), and (10) a plasticizer (e.g., oleyl alcohol and/or petrolatum).

Alternatively, the skin care composition comprising the siloxane-urethane-urea copolymer and the carrier, described above, may be an antiperspirant composition comprising: (1) the siloxane-urethane-urea copolymer described above, (2) a wax and/or thickener (e.g., beeswax, sodium stearate, stearyl alcohol, and/or carnauba), (3) an emollient (e.g. mineral oil, cyclomethicone, propylene glycol, and/or alcohol, (4) a fixative (e.g., silicone MQ or copolymer type resins and/or acrylate copolymers), (5) an antiperspirant active (e.g. aluminum chlorohydrate), and (6) a fragrance and/or perfume.

Alternatively, the skin care composition comprising the siloxane-urethane-urea copolymer and the carrier, described above, may be a sunscreen composition comprising: (1) the siloxane-urethane-urea copolymer described above, (2) an organic or physical sun blocker (e.g., oxybenzone, octocrylene, titanium dioxide, zinc oxide, and/or avobenzone), (3) a preservatives (e.g., phenoxyethanol), (4) an emulsifier and/or surfactant, (5) an emollient, (6) a thickener, (7) water, and (8) a moisturizer (e.g., glycerin).

Exemplary hair care compositions that can be made with the composition comprising copolymer (A) and the carrier, described above, include a shampoo comprising: (1) copolymer (A) described above, or the emulsion of copolymer (A) described above, (2) water, and (3) an anionic surfactant and/or an amphoteric surfactant (e.g., sodium laureth sulfate), optionally (4) a preservative, and optionally (5) a deposition agent (e.g., a cationic deposition polymer), and optionally (6) a thickener (e.g., carbomer).

Alternatively, the hair care product may be a hair conditioner comprising: (A) copolymer (A) described above, or the emulsion of copolymer (A) described above, (B) water, optionally (C) a thickener (e.g., hydroxyethyl-cellulose), (D) a fatty alcohol (e.g., Cetearyl Alcohol), optionally (E) other emulsifiers (e.g., PEG-100 Stearate & Glyceryl Stearate), optionally (F) a preservative, and optionally (G) other conditioning agents (e.g., cationic surfactants and/or cationic polymers).

Alternatively, the composition comprising copolymer (A) and the carrier, described above, may be used as a leave-in or leave-on hair treatment composition.

The composition comprising copolymer (A) and a carrier, described above, can be used in a variety of personal care applications. In particular, said composition may be used in the personal care compositions disclosed in U.S. Pat. No. 6,051,216 to Barr et al.; U.S. Pat. No. 5,919,441 to Mendolia et al.; U.S. Pat. No. 5,981,680 to Petroff et al.; U.S. Patent Application 2010/0098648 to Yu, and WO 2004/060101 to Yu; in sunscreen compositions as disclosed in U.S. Pat. No. 6,916,464 to Hansenne et al.; in cosmetic compositions containing film-forming resins, as disclosed in U.S. Patent Application Publication 2003/0235552 to Yu; in the cosmetic compositions as disclosed in U.S. Patent Application Publication 2003/0235553 to Lu, U.S. Patent Application Publication 2003/0072730 to Tornilhac, U.S. Patent Application Publication 2003/0170188 to Ferrari et al., EP 1,266,647 to Tornilhac, EP 1,266,648 to Ferrari, et al., EP1,266,653 to Ferrari et al., WO2003/105789 to Lu, WO2004/000247 to Lu and WO2003/106614 to Lu; as additional agents to those disclosed in WO2004/054523 to Tournilhac; in long wearing cosmetic compositions as disclosed in U.S. Patent Application Publication 2004/0180032; and in transparent or translucent care and/or make up compositions as discussed in WO 2004/054524. The polyurethane-polyorganosiloxane copolymer is useful as a film forming agent in various applications, such as the personal care compositions described above. Alternatively, the polyurethane-polyorganosiloxane copolymer is useful in addition to, or instead of, the silicone acrylate in the personal care compositions in, for example, “A Second Generation Silicone Acrylate for use in Beauty Care Applications, IP.com Number: 000239161 Electronic Publication Date Oct. 17, 2014. The polyurethane-polyorganosiloxane copolymer is useful as a film forming agent in addition to, or instead of, the phenylsilsequioxane resin in, for example, “Personal Care Applications for Phenylsilsesquioxane Resins,” IP.com Number IPCOM000248667D, Electronic Publication Date Dec. 22, 2016. These references disclose various color cosmetic formulations, such as foundations, blushes, lipsticks, lip glosses, mascaras, eye shadows, eyebrow gels and eyeliners; skin care compositions such as sun care formulations, antiperspirants and deodorants; and hair care formulations such as shampoos, hair conditioners (leave on or rinse off) and hair waxes.

### EXAMPLES

These examples are intended to illustrate the invention to one of ordinary skill in the art and should not be interpreted as limiting the scope of the invention set forth in the claims. All measurements and experiments were conducted at 23° C., unless indicated otherwise. Abbreviations used in the specification are in Table 1, below.

TABLE 1
Abbreviations-

| Abbreviation | Meaning |
|---|---|
| AA | Allyl amine from Aldrich |
| AOH | Allyl alcohol |
| BD | 1,4-butanediol |
| DA | Diallyl amine from TCI |
| DMA | N, N-dimethylacetamide |
| EtAc | Ethyl acetate, from Sigma-Aldrich. Anhydrous for polymerization reaction. |
| | HPLC grade for dissolving and processing copolymers. |
| HDI | Hexam ethylene diisocyanate from Acros |
| IPDI | Isophorone diisocyanate from Alfa Aesar |
| TDI | Toluene Diisocyanate |
| THF | Tetrahydrofuran |
| C16 | Carbinol terminated polydimethylsiloxane having MW of 920 to 924 from |
| | Gelest, Product DMS-C16 |
| C21 | Carbinol terminated polydimethylsiloxane having MW of 4330 to 4680 from |
| | Gelest, Product DMS-C21 |
| C23 | Carbinol terminated polydimethylsiloxane having MW of 12000 from Gelest, |
| | Product DMS-C23 |
| C62 | Carbinol terminated polydimethylsiloxane with a molecular weight MW of |
| | 1670 from Dow Corning |
| FTIR | Fourier Transform Infra-Red |
| A15 | Amino propyl terminated polydimethylsiloxane with a molecular weight Mn |
| | of 3000 from Gelest, Product DMS-A15 |
| NMR | Nuclear Magnetic Resonance |
| ml | Milliliters |
| ° C. | Degrees Celsius |
| Mn | Number average molecular weight determined by NMR |
| NMR | Nuclear magnetic resonance |

### Reference Example 1: General Procedure for Preparing Copolymer

A ml 4 neck flask was placed into a temperature controlled heating block and fitted with mechanical stirrer, thermometer, dropping funnel and reflux condenser.

1) The flask was charged with an a) isocyanate compound and a b) polyorganosiloxane, which were mixed to form a mixture.

2) The mixture was stirred and heated at 60° C., and the progress of the reaction followed by FTIR.

Optionally 3) After a period of time, solvent was added and the reaction cooled to room temperature of 15° C. to 40° C.

4) An c) endblocker (and optionally e) solvent) were charged to the dropping funnel and added drop-wise to the mixture in the flask, which was then heated for a period of time.

5) The mixture in the flask was cooled to room temperature and filtered through a 0.45 micron filter using Celite® 545 filter aid. The filtrate was transferred into a round flask and volatiles removed with a rotary evaporator (90° C., 1 mbar).

Copolymers were prepared according to this procedure using starting materials and conditions shown in Table 2 and labeled as Reference Example 1. The copolymers were characterized, and results are in Table 3.

### Example 2: Foundation Composition

A foundation composition was prepared by mixing the ingredients in Table 4, as follows. The ingredients in phase A were mixed until homogenous, the ingredients in phase B were mixed until homogenous, and the ingredients in phase C were mixed using a high shear mixer to form a dispersion. Next, phase C was added to phase A. This was mixed while adding phase B slowly with a high rate of agitation.

TABLE 4
Foundation Composition Preparation using the Copolymers
prepared in Reference Example 1

| | Copolymer Sample | | Reference |
|---|---|---|---|
| Copolymer solution | Solids content: | | Example 1 |
| concentration by weight | Trade Name/ | | 50.00% |
| Ingredient | Supplier | Wt. % | Grams |
| Phase A | | | |
| Emulsifier Blend | ES-5300 | 6 | 1.20 |
| | Formulation | | |
| | Aid from | | |
| | Dow Corning | | |
| | Corporation | | |
| Isododecane | — | q.s. | 1.40 |
| Film-Former | — | 5 | 2.00 |
| (% copolymer | | | |
| in the foundation) | | | |
| Phase B | | | |
| Water | — | 59.5 | 11.9 |
| Sodium Chloride | Fisher | 1 | 0.2 |
| Glycerin | Fisher | 5 | 1 |
| Phenoxyethanol, | Euxyl ® PE | 1 | 0.2 |
| Ethylhexylglycerin | 9010/Schülke & | | |
| | Mayr GmbH | | |
| Phase C | | | |
| Iron Oxide (CI77499), | SA-C335000- | 0.07 | 0.01 |
| Dimethicone | 10/Miyoshi | | |
| | Europe S.A.S. | | |
| Iron Oxide (CI77491), | SA-C332199- | 0.25 | 0.05 |
| Dimethicone | 10/Miyoshi | | |
| | Europe S.A.S. | | |
| Iron Oxide (CI77492), | SA-C331700- | 1.09 | 0.22 |
| Dimethicone | 10/Miyoshi | | |
| | Europe S.A.S. | | |
| Titanium Dioxide, | SA-TAO- | 5.81 | 1.16 |
| Dimethicone | 77891/Miyoshi | | |
| | Europe S.A.S. | | |
| Caprylyl Methicone | Dow Corning ® | 3.28 | 0.66 |
| | FZ-3196 | | |
| | Volatile Alkyl | | |
| | Methyl Siloxane | | |
| | Fluid | | |
| | | total (g): | 20.00 |

### Example 3 (Comparative)

Comparison samples were prepared by either taking away the copolymer and replacing it with isododecane (Control in table 6), or by replacing the copolymer with a comparative film former, which was an MQ resin produced by Dow Corning Corporation commercially being used as a film former in similar types of formulations (MQ Resin (749) in Table 6).

### Example 4—Evaluation of Samples prepared in Examples 2 and 3

Hydrated collagen substrate samples were prepared by pulling hydrated collagen over polycarbonate blocks. The foundation compositions described above were coated onto the hydrated collagen using a 2 micrometer film gauge. The resulting coating had a thickness of 150 micrometers. Each foundation composition was allowed to dry overnight to form a foundation on the hydrated collagen. Using a Gardner abrasion tester fitted with an adhesive strip of the loop side of Velcro, each sample was insulted for either 50 or 20 cycles, depending on whether the film was dry or sebum was added. Three blocks were tested per foundation. Each foundation was tested dry, however some of the best performers were tested for sebum resistance which required adding a drop of artificial sebum to the foundation, and evenly spreading it out; and then waiting a minute before beginning the abrasion testing.

A Hunterlab colorimeter was used to measure the LAB (color) value for each block using 0.5″ diameter filter. Measurements were taken at 0, 4, 8, 12, 16, 20, 30, 40, and 50 insults. If using sebum the testing stopped at 20 insults. From this a ΔE was calculated to determine the total change in color.

TABLE 6
Abrasion resistance test results, as measured
by the amount of color changes.

| # | | | |
|---|---|---|---|
| of | ΔE | | |
| Abrasion | | MQ Resin | Copolymer of |
| Cycles | Control | (749) | Reference Example 1 |
| 0 | 0 | 0 | |
| 4 | 7.94 | 3.56 | |
| 8 | 9.42 | 3.79 | |
| 12 | 10.28 | 3.80 | |
| 16 | 10.81 | 3.99 | |
| 20 | 11.11 | 4.17 | |
| 30 | 11.67 | 4.52 | |
| 40 | 12.00 | 4.88 | |
| 50 | 12.18 | 5.06 | |

Contact angle was measured on the samples by diluting the copolymers prepared in Reference Example 1 in ethyl acetate, to form solutions that were 80% ethyl acetate and 20% copolymer. The solutions were then coated onto cleaned glass microscope slides using a 50 micrometer film gauge. Three slides were made for each sample, and the slides were allowed to dry overnight. A VCA Optima device was used to test the slides for both water and artificial sebum. For water, 2 microns of water was added to slides with each sample dried thereof. For sebum, 1 microliter of artificial sebum was added. One contact angle measurement was taken per slide for each water and artificial sebum.

Solution samples were prepared by diluting the copolymers prepared in Reference Example 1 in ethyl acetate, to form solutions that were 80% ethyl acetate and 20% copolymer. The solution samples were coated onto 10 centimeter long latex elastic bands using a 50 micrometer film gauge. The latex bands were 0.33 millimeter thick specimens from Four D Rubber Co. Ltd. in United Kingdom, Red Latex Rubber: incolore solution. One band was coated per sample, and coated bands were allowed to dry overnight. Photos were taken the next day before any measurements. Films were then stretched to 200% of their original length (20 cm). Photos were taken again to capture any cracking or differences in shine.

Example 4 shows that the foundation prepared from the personal care composition of this invention, which includes a polyurethane-polyorganosiloxane copolymer, has superior abrasion resistance to a foundation prepared using a composition with a conventional MQ resin film forming agent. Additional benefits include improved flexibility for comfort of wear, and shine.

### Example 5: Preparation of Emulsions (Prophetic)

The emulsification procedure will be performed on a Hauschild Speedmixer. To a plastic cup will be added polyurethane-polyorganosiloxane copolymer prepared in Reference Example 1, Brig 35L surfactant, optionally lactic acid, and a first quantity of water. The mixture will be sheared at maximum speed for 20 seconds. An additional quantity of water will be added, and the emulsion will again be mixed on the Speedmixer. The amounts in each emulsion sample to be prepared are shown in Table 6, below.

TABLE 6
Ingredients for preparing emulsions, weight parts

| | Amount in | Amount in | Amount in | Amount in |
|---|---|---|---|---|
| Ingredient | Sample A | Sample B | Sample C | Sample D |
| polyurethane- | 10 | 4 | 10 | 4 |
| polyorganosiloxane | | | | |
| copolymer | | | | |
| Brig 35L Surfactant | 0.5 | 4 | 0.6 | 4 |
| Water, First | 3 + 3 | 2 + 8 | 2 + 4 | 2 + 7 |
| Quantity + | | | | |
| Additional Quantity | | | | |
| Lactic Acid | none | 0.1 | none | 0.06 |

### Example 6: Preparation of Hair Conditioner Formulations (Prophetic)

Samples of emulsions as described in Example 5 will be added to rinse-off conditioning formulations using an amount sufficient to provide 2% the silicone block copolymer. The conditioning formulations are shown in Table 2, below. The conditioners of the present invention will be prepared using Emulsions A, B, C and D from Table 6.

TABLE 7
Conditioners

| Ingredient | Weight % | Weight % | Weight % | Weight % |
|---|---|---|---|---|
| Deionized Water | q.s. to | q.s. to | q.s. to | q.s. to |
| | 100% | 100% | 100% | 100% |
| Hydroxyethyl-cellulose¹ | 1.5 | 1.5 | 1.5 | 1.5 |
| Cetearyl Alcohol² | 1.0 | 1.0 | 1.0 | 1.0 |
| PEG-100 Stearate & Glyceryl Stearate³ | 1.0 | 1.0 | 1.0 | 1.0 |
| Emulsion A | 3.3 | — | — | — |
| Emulsion B | — | 10.0 | — | — |
| Emulsion C | — | — | 3.4 | — |
| Emulsion D | — | — | — | 9.6 |
| Phenoxyethanol and Ethylhexylglycerin⁴ | 0.5 | 0.5 | 0.5 | 0.5 |

¹Natrosol ® 250 HHR available from Hercules of Wilmington, DE
²Crodocol CS-50 ® available from Croda Inc. of Edison, NJ
³Arlacel ® 165 available from Uniqema of Wilmington, DE
⁴Euxyl ® PE 9010 available from Schülke & Mayr

### Example 8—Conditioning Shampoo Formulations (Prophetic)

Samples of emulsions described in Example 5 will be added to shampoo formulations in an amount sufficient to provide 2% of the polyorganosiloxane-polyurethane copolymer. The shampoo formulations are shown in Table 8. The shampoos of the present invention will be prepared using A, B, C and D emulsions from Table 6.

TABLE 8
Conditioning Shampoos

| | Weight % | Weight % | Weight % | Weight % |
|---|---|---|---|---|
| Ingredient | q.s. to | q.s. to | q.s. to | q.s. to |
| Deionized Water | 100% | 100% | 100% | 100% |
| Sodium Laureth Sulfate¹ | 30 | 30 | 30 | 30 |
| Cocamide MIPA² | 2.0 | 2.0 | 2.0 | 2.0 |
| Cocamidopropyl Betaine³ | 7.0 | 7.0 | 7.0 | 7.0 |
| Polyquaternium-10⁴ | 0.3 | 0.3 | 0.3 | 0.3 |
| PEG-150 Pentaerythrityl | 1.5 | 1.5 | 1.5 | 1.5 |
| Tetrastearate⁵ | | | | |
| Emulsion A | 3.3 | — | — | — |
| Emulsion B | — | 10.0 | — | — |
| Emulsion C | — | — | 3.4 | — |
| Emulsion D | — | — | — | 9.6 |
| Phenoxyethanol (and) | 0.5 | 0.5 | 0.5 | 0.5 |
| Ethylhexylglycerin⁶ | | | | |

¹Standapol ES-3 ® available from Cognis Corp. of Cincinnati, OH
²Mackamide CPA available from Rhodia3. Monateric CAB-LC ® available from Uniquema of New Castle, DE
⁴UCARE Polymer JR-30M available from Dow/Amerchol of Midland, MI
⁵Crothix ® available from Croda Inc. of Edison, NJ
⁶Euxyle ® PE 9010 available from Schülke & Mayr

Deionized water is added to the mixing vessel. In order to keep the active silicone loading constant throughout testing, the water level may be adjusted by adding water depending on the percent copolymer in the various emulsions used. With moderate agitation, the polyquaternium-10 is dispersed until fully dissolved. This is then heated to 75° C. and the PEG-150 pentaerythrityl tetrastearate is added with continual mixing. Heat is decreased to 40° C. and sodium lauryl ether sulfate, cocamide MIPA, cocamidopropyl betaine are added in that order. When completely incorporated, polyurethane-polyorganosiloxane copolymer emulsion is added to the base shampoo. The shampoo is mixed for 5-10 minutes and then Phenoxyethanol (and) Ethylhexylglycerin is added. The water loss is compensated for and the formulation is mixed for an additional 5 minutes. The final pH of the shampoo formulations are approximately 5.5-6.0.

All amounts, ratios, and percentages are by weight unless otherwise indicated. The articles ‘a’, ‘an’, and ‘the’ each refer to one or more, unless otherwise indicated. The disclosure of ranges includes the range itself and also anything subsumed therein, as well as endpoints. For example, disclosure of a range of 2.0 to 4.0 includes not only the range of 2.0 to 4.0, but also 2.1, 2.3, 3.4, 3.5, and 4.0 individually, as well as any other number subsumed in the range. Furthermore, disclosure of a range of, for example, 2.0 to 4.0 includes the subsets of, for example, 2.1 to 3.5, 2.3 to 3.4, 2.6 to 3.7, and 3.8 to 4.0, as well as any other subset subsumed in the range. Similarly, the disclosure of Markush groups includes the entire group and also any individual members and subgroups subsumed therein. For example, disclosure of the Markush group a hydrogen atom, an alkyl group, an aryl group, or an aralkyl group includes the member alkyl individually; the subgroup alkyl and aryl; and any other individual member and subgroup subsumed therein.

“Alkyl” means a saturated monovalent hydrocarbon group. Alkyl is exemplified by, but not limited to, methyl, ethyl, propyl (e.g., iso-propyl and/or n-propyl), butyl (e.g., isobutyl, n-butyl, tert-butyl, and/or sec-butyl), pentyl (e.g., isopentyl, neopentyl, and/or tert-pentyl); hexyl, heptyl, octyl, nonyl, and decyl, as well as branched saturated monovalent hydrocarbon groups of 6 or more carbon atoms.

“Alkenyl” means a monovalent hydrocarbon group containing a double bond. Alkenyl groups are exemplified by, but not limited to, ethenyl, propenyl (e.g., iso-propenyl and/or n-propenyl), butenyl (e.g., isobutenyl, n-butenyl, tert-butenyl, and/or sec-butenyl), pentenyl (e.g., isopentenyl, n-pentenyl, and/or tert-pentenyl), hexenyl, heptenyl, octenyl, nonenyl, and decenyl, as well as such branched groups of 6 or more carbon atoms.

“Alkynyl” means a monovalent hydrocarbon group containing a triple bond. Alkynyl groups are exemplified by, but not limited to, ethynyl, propynyl (e.g., iso-propynyl and/or n-propynyl), butynyl (e.g., isobutynyl, n-butynyl, tert-butynyl, and/or sec-butynyl), pentynyl (e.g., isopentynyl, n-pentynyl, and/or tert-pentynyl), hexynyl, heptynyl, octynyl, nonynyl, and decynyl, as well as such branched groups of 6 or more carbon atoms.

“Aryl” means a cyclic, fully unsaturated, hydrocarbon group. Aryl is exemplified by, but not limited to, cyclopentadienyl, phenyl, anthracenyl, and naphthyl. Monocyclic aryl groups may have 5 to 9 carbon atoms, alternatively 6 to 7 carbon atoms, and alternatively 5 to 6 carbon atoms. Polycyclic aryl groups may have 10 to 18 carbon atoms, alternatively 10 to 14 carbon atoms, and alternatively 12 to 14 carbon atoms.

“Aralkyl” means an alkyl group having a pendant and/or terminal aryl group or an aryl group having a pendant alkyl group. Exemplary aralkyl groups include tolyl, xylyl, benzyl, phenylethyl, phenyl propyl, and phenyl butyl.

“Carbocycle” and “carbocyclic” each mean a hydrocarbon ring. Carbocycles may be monocyclic or alternatively may be fused, bridged, or spiro polycyclic rings. Monocyclic carbocycles may have 3 to 9 carbon atoms, alternatively 4 to 7 carbon atoms, and alternatively 5 to 6 carbon atoms. Polycyclic carbocycles may have 7 to 18 carbon atoms, alternatively 7 to 14 carbon atoms, and alternatively 9 to 10 carbon atoms. Carbocycles may be saturated or partially unsaturated.

“Cycloalkyl” means saturated carbocycle. Monocyclic cycloalkyl groups are exemplified by cyclobutyl, cyclopentyl, and cyclohexyl.

Collectively, the term “monovalent hydrocarbon group” includes alkyl, alkenyl, aryl, aralkyl, and carbocyclic groups, as defined above.

“Divalent hydrocarbon group” includes alkylene groups such as ethylene, propylene (including isopropylene and n-propylene), and butylene (including n-butylene, t-butylene and isobutylene); and pentylene, hexylene, heptylene, octylene, and branched and linear isomers thereof; arylene groups such as phenylene; and alkaralkylene groups such as:

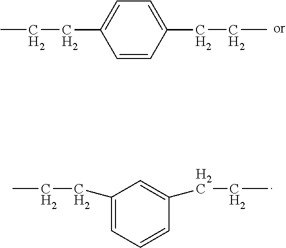

Alternatively, each divalent hydrocarbon group may be ethylene, propylene, butylene or hexylene. Alternatively, each divalent hydrocarbon group may be ethylene or propylene.

“Halogenated hydrocarbon” means a hydrocarbon group as defined above, but where one or more hydrogen atoms bonded to a carbon atom have been formally replaced with a halogen atom. For example, monovalent halogenated hydrocarbon groups can be any one of alkyl, alkenyl, aryl, aralkyl, and carbocyclic groups in which one or more hydrogen atoms bonded to a carbon atom have been replaced with a halogen atom. Monovalent halogenated hydrocarbon groups include haloalkyl groups, halogenated carbocyclic groups, and haloalkenyl groups. Haloalkyl groups include fluorinated alkyl groups such as trifluoromethyl (CF₃), fluoromethyl, trifluoroethyl, 2-fluoropropyl, 3,3,3-trifluoropropyl, 4,4,4-trifluorobutyl, 4,4,4,3,3-pentafluorobutyl, 5,5,5,4,4,3,3-heptafluoropentyl, 6,6,6,5,5,4,4,3,3-nonafluorohexyl, and 8,8,8,7,7-pentafluorooctyl; and chlorinated alkyl groups such as chloromethyl and 3-chloropropyl. Halogenated carbocyclic groups include fluorinated cycloalkyl groups such as 2,2-difluorocyclopropyl, 2,3-difluorocyclobutyl, 3,4-difluorocyclohexyl, and 3,4-difluoro-5-methylcycloheptyl; and chlorinated cycloalkyl groups such as 2,2-dichlorocyclopropyl, 2,3-dichlorocyclopentyl. Haloalkenyl groups include chloro allyl.

“Skin” includes stratum corneum covered skin and mucosal membranes.

1. A personal care composition comprising:
(1) a siloxane-urethane-urea copolymer (A) of unit formula 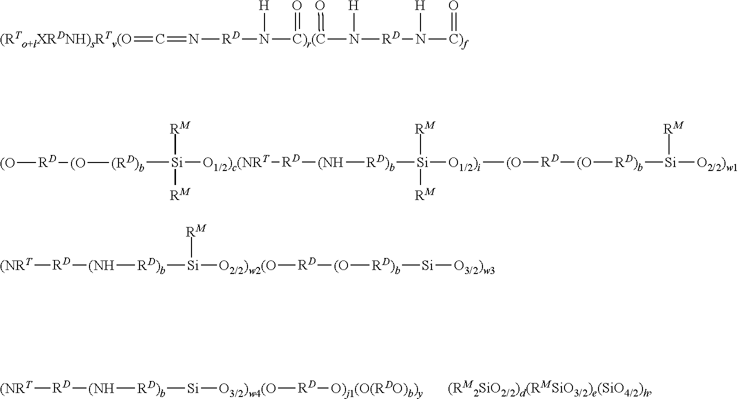
where
each R^{D} is independently a divalent hydrocarbon group a divalent halogenated hydrocarbon group;
each R^{M} is independently a monovalent hydrocarbon group or a monovalent halogenated hydrocarbon group;
each R^{T} is independently hydrogen or a hydrocarbon group;
each subscript b is independently 0 to 1,000,000;
subscript c is 0 to to 200,000, subscript i is 0 to 200,000, subscript w1 is 0 to 200,000, subscript w2 is 0 to 200,000, subscript w3 is 0 to 200,000, subscript w4 is 0 to 200,000, and a quantity (c+i+w1+w2+w3+w4) is 1;
subscript d is 0 to 1,000,000;
subscript e is 0 to 1,000,000;
subscript f is 0 to 1,500,000;
subscript h is 0;
subscript j1 is 1;
each X is independently nitrogen, oxygen, or sulfur;
subscript o=0 when X is oxygen or sulfur, and subscript o=1 when X is nitrogen;
subscript r is 0 to 1,500,000, and the quantity f+r is ≥1;
subscript s is 0 to 200,000; and
subscript vis 0 to 200,000;
subscript y is ≥0;
and
(2) a carrier that permits application; and
(3) a silicone.

2. The composition of claim 1, where the composition further comprises one or both of starting materials (B) and (C), where
starting material (B) is an organic polyol; and
starting material (C) is a reaction product of an organic polyisocyanate and an organic polyol.

3. The composition of claim 1, where the composition is selected from a solution or a dispersion.

4. The composition of claim 1, where the composition is an emulsion, and the carrier comprises water and a surfactant.

5. The composition of claim 1, where copolymer (A) comprises unit formula (I): 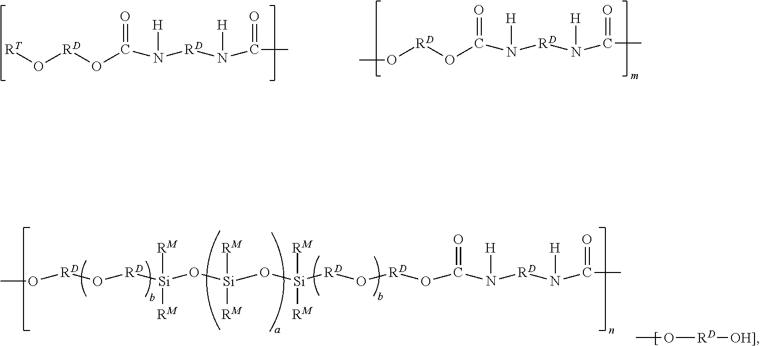
where each subscript a is independently 0 to 1,000,000; each subscript b is independently greater than or equal to 0; each subscript m is ≥1, and subscript n is greater than or equal to 1.

6. The composition of claim[[s]] 1 to 5, where copolymer (A) comprises unit formula (III): 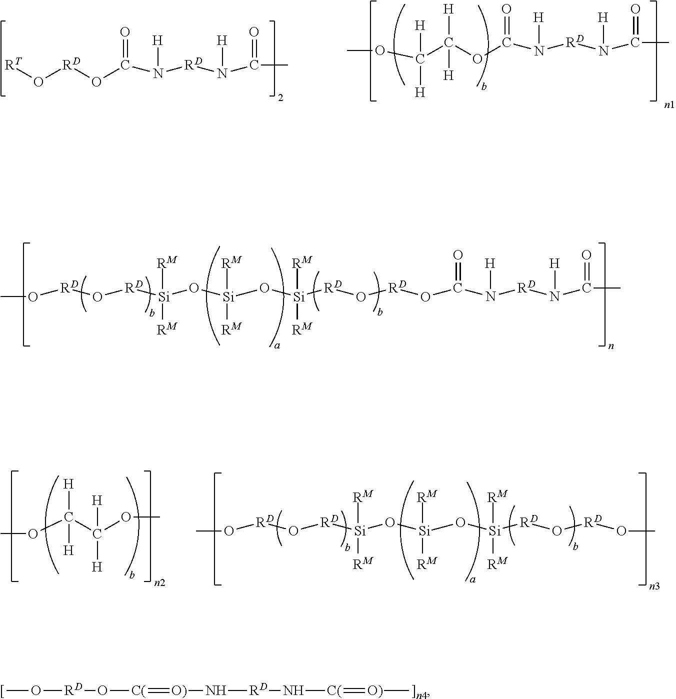
where subscript n is greater than or equal to 1, subscript n1 is greater than or equal to 0, subscripts n2 and n3 are 0 or 1, and a quantity (n2+n3)=1, and subscript n4 is greater than or equal

7. A method comprising:
(1) applying the composition of claim 1 to a substrate, and
(2) drying the substrate.

8. The method of claim 7, where the substrate is selected from the group consisting of human skin and human hair.

9.-11. (canceled)

12. The composition of claim 2, where the composition is selected from a solution or a dispersion.

13. The composition of claim 2, where the composition is an emulsion, and the carrier comprises water and a surfactant.

14. The composition of claim 2, where copolymer (A) comprises unit formula (I): 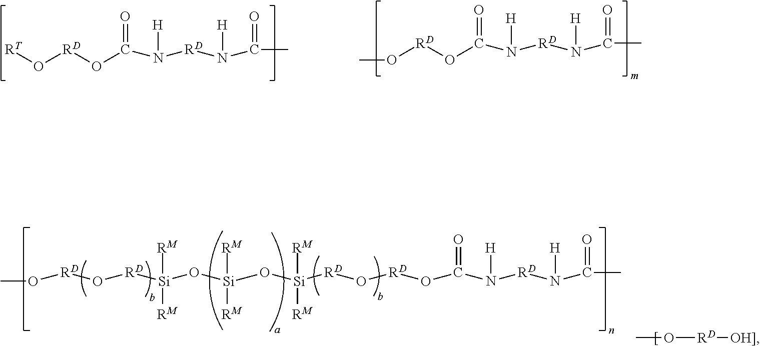
where each subscript a is independently 0 to 1,000,000; each subscript b is independently greater than or equal to 0; each subscript m is ≥1, and subscript n is greater than or equal to 1.

15. The composition of claim 2, where copolymer (A) comprises unit formula (III): 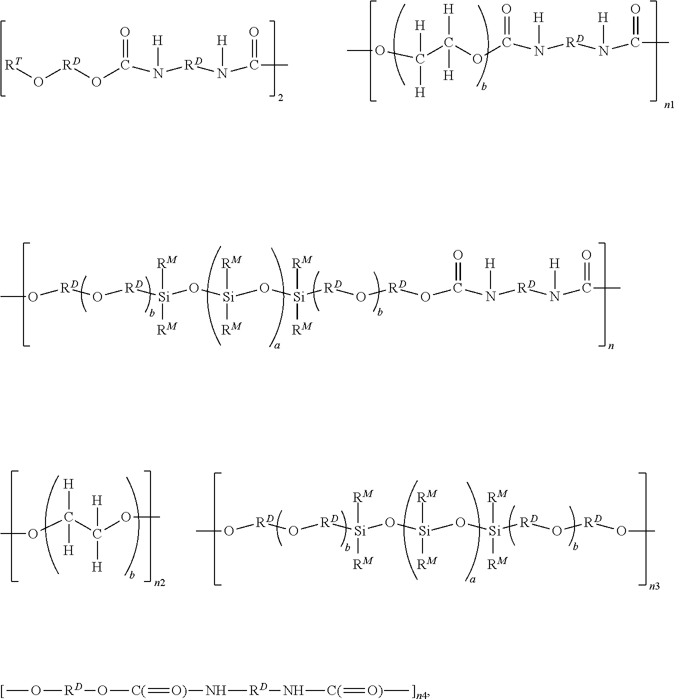
where subscript n is greater than or equal to 1, subscript n1 is greater than or equal to 0, subscripts n2 and n3 are 0 or 1, and a quantity (n2+n3)=1, and subscript n4 is greater than or equal 1.

16. The composition of claim 1, where the silicone is selected from the group consisting of fluids, gums, resins, elastomers, surfactants, alkylmethylsilicones, and/or silicone carbinol fluids.
